(19) United States
(12) Patent Application Publication
(10) Pub. No.: US 2011/0042211 A1
(43) Pub. Date: Feb. 24, 2011

(54) BIOCHEMICAL TEST STRIP, MEASUREMENT DEVICE, AND BIOCHEMICAL TEST SYSTEM

(75) Inventors: Ying-Che HUANG, Jhonghe City (TW); Mon Wen YANG, Hsinchu City (TW); Thomas Y.S. SHEN, Hsinchu City (TW)

Correspondence Address: LIU & LIU, 444 S. FLOWER STREET SUITE 1750, LOS ANGELES, CA, 90071, US

(73) Assignee: APEX BIOTECHNOLOGY CORP., Hsinchu City (TW)

(21) Appl. No.: 12/785,369

(22) Filed: May 21, 2010

(30) Foreign Application Priority Data
Aug. 21, 2009 (TW) 98215494

(51) Int. Cl. G01N 27/26; C12Q 1/26

(52) U.S. Cl. 204/403.04; 204/403.02

(57) ABSTRACT

A biochemical test strip, a measurement device, and a biochemical test system are provided. The biochemical test strip has a first connection region, a second connection region and a sensing region defined thereon, and includes an insulating substrate, a set of electrodes, an insulating slice and an identifying unit. The set of the electrodes is disposed on the insulating substrate, and one end of the set of electrodes is in the first connection region. The insulating slice is disposed on the set of the electrodes and exposes at least the first connection region. The identifying unit including a plurality of electronic elements is formed on a surface of the insulating slice in the second connection region, wherein the second connection region is different from the first connection region. The type of the biochemical test strip is determined by the number and location of the plurality of electronic elements.

### CROSS REFERENCE TO RELATED APPLICATIONS

This application claims priority to Taiwan Patent Application No. 98215494 entitled. “BIOCHEMICAL TEST STRIP, MEASUREMENT DEVICE, AND BIOCHEMICAL TEST SYSTEM”, filed on Aug. 21, 2009, which is incorporated herein by reference and assigned to the assignee herein.

### FIELD OF INVENTION

The present invention relates to a biochemical test strip, a measurement device, and a biochemical test system, and more particularly, to a biochemical test strip having self-identification function, a measurement device for use with the same, and a biochemical test system having the same.

### BACKGROUND OF THE INVENTION

The self-testing products, such as the biochemical test strips, can be used for biochemical analysis of body fluid, including the measurement of blood sugar, lactic acid, cholesterol, etc. However, the characteristics of the biochemical test strips may vary from batch to batch due to the process variations. Therefore, most biochemical test strips need to be calibrated or identified by a code card.

For example, U.S. Pat. No. 5,366,609 and PCT patent No. WO00/33072 disclose a biosensing meter provided with a pluggable read-only memory (ROM), i.e. the code card. The pluggable code card includes a plurality of stored parameter values for calibrating a measurement device. However, the manufacture of the code card will increase the production cost and the labor power, and besides, the correction errors and the data measurement errors occur frequently because users may forget to insert the code card, use a wrong code card, or lose the code card.

To solve the inconvenience associated with using the code card, U.S. Pat. No. 6,814,844 discloses an identification method using bar code. The bar code pattern is formed by using a high-energy pulsed laser to bombard a surface of a gold target material coated on the substrate, so as to remove a portion of the gold target material. However, as disclosed in U.S. Pat. No. 6,814,844, the bar code is detected by optical detecting method, such as CCD or LED. Moreover, the reproduction and the accuracy of the bar code highly depend on the surface condition of the target material, and therefore there is not only a limitation to the fabrication, but also an increase in the production cost.

In addition, Taiwan utility model patent No. M304662 discloses a biochemical test system capable of being exempted from using a code card. The measurement device is equipped with several buttons which allow a user to enter specific English characters or numbers corresponding to a set of parameters stored in a correction unit of the measurement device. These characters or numbers may be printed on the exterior package of the test strip (packing case, plastic box, manual, etc.). After entering the specific English characters or numbers, a microprocessor of the measurement device can select corresponding correction parameters to calibrate the measurement device.

Further, Taiwan patent application No. 97208206 discloses a test strip capable of avoiding the need of the code card. A plurality of identifying elements are formed on one end of the test strip, and each identifying element can be punched selectively to construct various code patterns. However, there are a lot of limitations in this test strip, such as high precision requirement of punching process, high accuracy requirement of alignment between the sensing terminals of a measurement device and the identifying elements of the test strip, and risk of breaking the test strip due to its tooth-like shape.

For obviating the problems of high cost, complicated process, and/or inconvenience in operation, it is advantageous to have a biochemical test strip capable of being calibrated without the code card correction and providing easy operation to users.

### SUMMARY OF THE INVENTION

In view of the problems existing in the prior arts, the present invention provides a biochemical test system, a measurement device, and a biochemical test strip capable of providing self-identification function, eliminating the use of a discrete code card, and reducing the possibility of man-made errors, and increasing operating convenience.

According to an aspect of the present invention, a biochemical test strip having a first connection region, a second connection region and a sensing region defined thereon is provided, wherein the second connection region is different from the first connection region. The biochemical test strip includes an insulating substrate, a set of electrodes, an insulating slice and an identifying unit. The set of electrodes is disposed on the insulating substrate and one end of the set of electrodes is in the first connection region. The insulating slice is disposed on the set of the electrodes and exposes at least the first connection region. The identifying unit having a plurality of electronic elements is formed on a surface of the insulating slice in the second connection region. The identification code of the biochemical test strip is determined by number and location of the plurality of electronic elements.

According to another aspect of the present invention, a biochemical test strip having a first connection region, a second connection region and a sensing region defined thereon is provided, wherein the second connection region is different from the first connection region. The biochemical test strip includes an insulating substrate having an upper surface, a set of electrodes, and an identifying unit. The set of electrodes is disposed on the insulating substrate, and one end of the set of electrodes is in the first connection region. The identifying unit is disposed in the second connection region and includes a plurality of electronic elements. The distance between the identifying unit and the upper surface is different from the distance between the set of electrodes and the upper surface. The identification code of the biochemical test strip is determined by number and location of the plurality of electronic elements.

According to another aspect of the present invention, a measurement device for used with the above-described biochemical test strip is provided, which includes a connector and a microprocessor. The connector includes a plurality of connecting terminals respectively corresponding to the set of electrodes and the identifying unit. The plurality of connecting terminals are electrically coupled to the set of electrodes and the identifying unit, and configured to receive a signal corresponding the identifying unit. The microprocessor is coupled to the connector for receiving the signal from the connector.

According to another aspect of the present invention, a measurement device for used with the above-described biochemical test strip is provided, which includes a connector configured to being electrically connected to the biochemical test strip and a microprocessor electrically coupled to the connector.

According to another aspect of the present invention, a biochemical test system including both of the above-described biochemical test strip and a measurement device is provided. The measurement device includes a microprocessor and a connector, wherein the connector includes a plurality of connecting terminals respectively corresponding to the set of electrodes and the identifying unit of the biochemical test strip. The plurality of connecting terminals are electrically coupled to the set of electrodes and the identifying unit for receiving a signal corresponding to the identifying unit. The microprocessor is coupled to the connector for receiving the signal from the connector.

The other aspects of the present invention, part of them will be described in the following description, part of them will be apparent from description, or can be known from the execution of the present invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE PICTURES

The foregoing aspects and many of the attendant advantages of this invention will become more readily appreciated as the same becomes better understood by reference to the following detailed description, when taken in conjunction with the accompanying pictures, wherein:

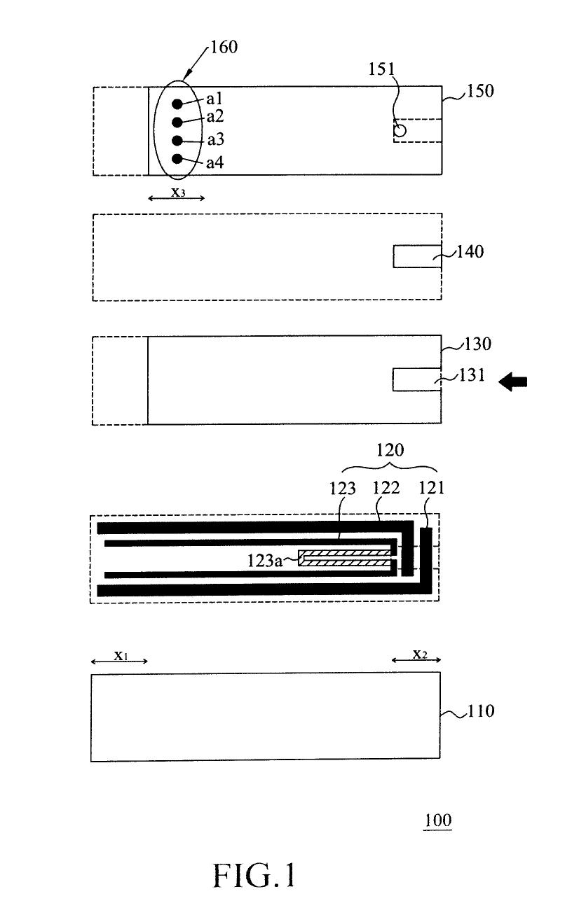

FIG. 1 illustrates an explosive view of a biochemical test strip according to an embodiment of the present invention;

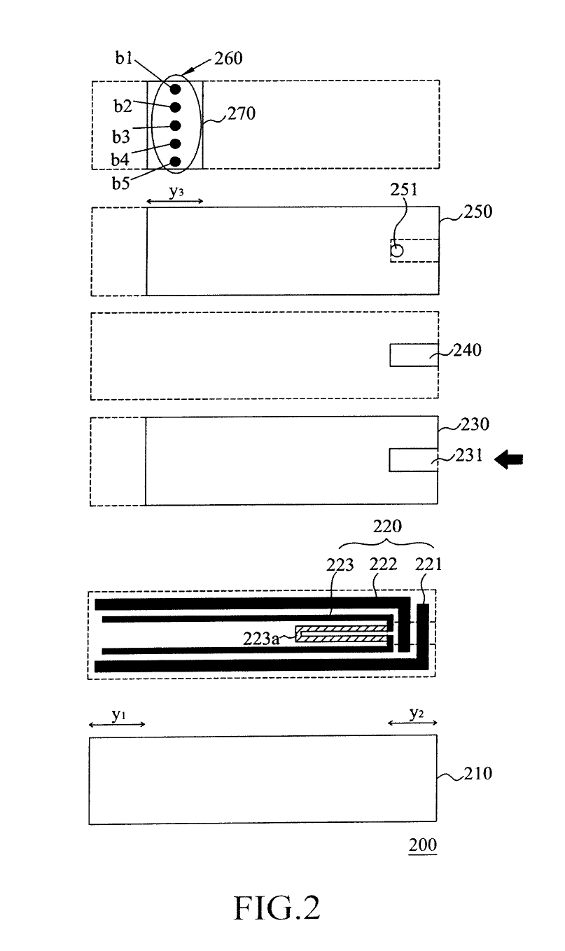

FIGS. 2-4 are the biochemical test strips according to different embodiments of the present invention respectively;

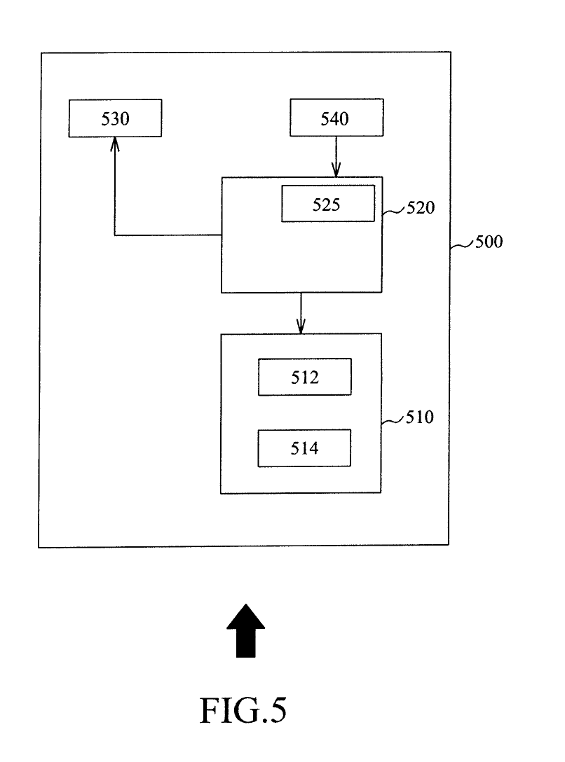

FIG. 5 is a block diagram of a measurement device according to an embodiment of the present invention; and

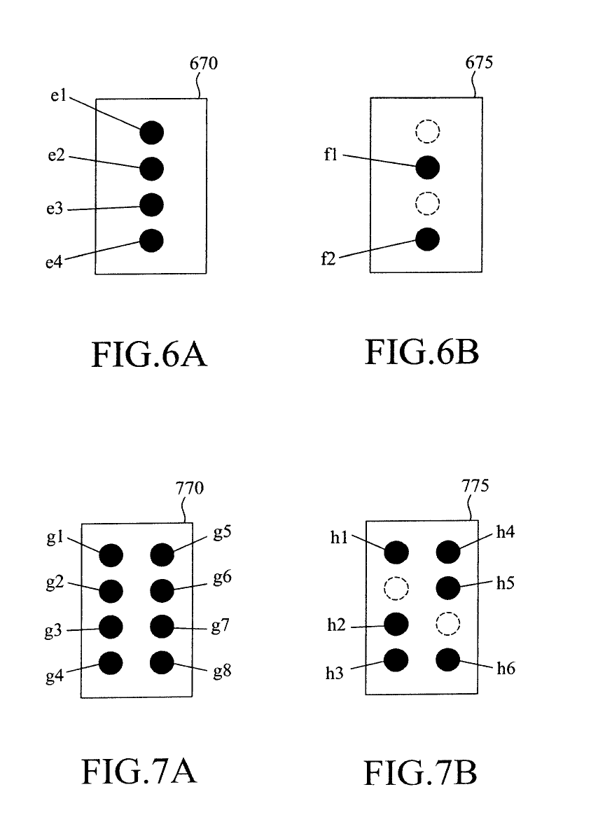

FIGS. 6A, 6B, 7A, 7B, 8, 9, 10A and 10B are illustrative diagrams showing the insulating slices and the identifying units thereon according to different embodiments of the present invention respectively.

### DETAILED DESCRIPTION OF THE INVENTION

The present invention discloses a biochemical test system, a measurement device, and a biochemical test strip, which can eliminate the need of a discrete code card, provide easy operation for the user, prevent from forgetting to insert the code card or using a wrong code card, and reducing the possibility of errors during the production process. The present invention will be described more fully hereinafter with reference to the FIGS. 1-10B. However, it should be noted that the features illustrated in the drawings are not necessarily drawn to scale, and like reference numerals represent the same or similar elements. The devices, elements, and methods in the following description are configured to illustrate the present invention, and should not be construed in a limiting sense. Furthermore, it should be noted that when an element is referred to as being “on” another element, it can be directly on the other element or intervening elements may also be present, unless explicitly defined otherwise herein.

FIG. 1 illustrates an explosive view of a biochemical test strip 100 according to an embodiment of the present invention, while the dotted line is used to indicate the relative position of each element. The biochemical test strip 100 of the present invention includes an insulating substrate 110, a set of electrodes 120, an insulating spacing layer 130, a cover 150, and an identifying unit 160. Further, a first connection region x₁, a sensing region x₂, and a second connection region x₃ can be defined in the biochemical test strip 100 based on their respective functions. The first connection region x₁ and the second connection region x₃ are configured to be connected to a measurement device (such as measurement device 500 shown in FIG. 5), and the sensing region x₂ is configured to form a space for accommodating a sample.

The insulating substrate 110 is electrically insulating and can be made of materials including, but not limited to, polyvinylchloride (PVC), glass fiber (FR-4), polyester, bakelite, polyethylene terephthalate (PET), Polycarbonate (PC), polypropylene (PP), polyethylene (PE), polystyrene (PS), ceramic material, etc.

The set of electrodes 120 can be any known conductive material, such as carbon paste, gold-silver paste, copper paste, carbon/silver paste, other similar material, or the combination thereof. In an embodiment, the set of electrodes 120 includes a conductive silver paste layer and a conductive carbon paste layer disposed on the conductive silver paste layer. Typically, the impedance of the conductive carbon paste layer is much larger than that of the conductive silver (or other metal) paste layer. The set of electrodes 120 includes a plurality of electrodes insulated from one another. In one embodiment, the set of electrodes 120 includes a working electrode 121, a reference electrode 122, and a sensing electrode 123 insulated from one another. Two ends of each of the working electrode 121 and the reference electrode 122 are respectively located in the first connection region x₁ and the sensing region x₂, for being connected to a measurement device (such as the measurement device 500 shown in FIG. 5) and a sample respectively. In the embodiment shown in FIG. 1, the sensing electrode 123 is composed of two electrodes and a resistor 123a with a fixed resistance value, and both of two ends of the sensing electrode 123 are located in the first connection region x₁. When the biochemical test strip 100 is inserted into the measurement device, a loop is formed between the sensing electrode 123 and the measurement device, so as to initiate the measurement device. After the measurement device is initiated, a resistance value between two ends of the sensing electrode 123 can be measured and compared with the resistance value of the resistor 123a to determine whether the electrochemical test strip 100 is inserted into the measurement device properly. It should be noted that the shape of the sensing electrode 123 could be arbitrary as long as it is capable of forming an electric loop with the measurement device. For example, the sensing electrode 123 can be a [symbol] -shaped electrode without the resistor 123a. Typically, as long as the electrodes can achieve the above-mentioned functions and are electrically insulated from one another, the present invention doesn't limit the arrangement and the number of the electrodes. Any additional electrodes can be added to accommodate various application needs.

The insulating spacing layer 130 is disposed on the set of electrodes 120, and includes an opening 131 located in the sensing region x₂ to expose a part of the set of electrodes 120. Typically, as long as part of the working electrode 121 and part of the reference electrode 122 can be exposed by the opening 131, the present invention is not limited to the shape of the opening 131. Besides, the first connection region x₁ can be exposed by the insulating spacing layer 130 so that one end of the set of electrodes 120 located in the first connection region x₁ can be electrically connected to a measurement device (such as the measurement device 500 shown in FIG. 5). In this embodiment, the insulating spacing layer 130 covers the second connection region x₃. The material of the insulating spacing layer 130 can include, but is not limited to, PVC insulating tape, PET insulating tape, thermal drying insulating paint or ultraviolet drying insulating paint. Regarding the manufacturing process, the insulating spacing layer 130 can be formed separately with a precut opening 131 and then disposed on the insulating substrate 110 and the set of electrodes 120. Alternatively, the insulating spacing layer 130 can be directly formed on part of the insulating substrate 110 and the set of electrodes 120 by a printing method, skipping areas of the opening 131 and the first connection region x₁.

The biochemical test strip 100 of the present invention further includes a reaction layer 140 disposed within the opening 131, which has the ability to identify a specified organic material or substance. Generally, the reaction layer 140 should at least cover part of the working electrode 121 and the reference electrode 122. Typically, for reacting with the sample, the reaction layer 140 can be implemented by an oxidoreductase or an electronic mediator (e.g. Ferrous material), but the present invention is not limited to this only. The following table shows some examples of the material of the reaction layer 140, which are respectively corresponding to different samples.

| | | Mediator | |
|---|---|---|---|
| Analyte | Enzymes | (Oxidized Form) | Additional Mediator |
| Glucose | Glucose Dehydrogenase | Ferricyanide | |
| | and Diaphorase | | |
| Glucose | Glucose-Dehydrogenase | Ferricyanide | |
| | (Quinoprotein) | | |
| Cholesterol | Cholesterol Esterase and | Ferricyanide | 2,6-Dimethyl-1,4- |
| | Cholesterol Oxidase | | Benzoquinone |
| | | | 2,5-Dichloro-1,4- |
| | | | Benzoquinone or |
| | | | Phenazine Ethosulfate |
| HDL | Cholesterol Esterase | Ferricyanide | 2,6-Dimethyl-1,4- |
| Cholesterol | and Cholesterol Oxidase | | Benzoquinone |
| | | | 2,5-Dichloro-1,4- |
| | | | Benzoquinone or |
| | | | Phenazine Ethosulfate |
| Triglycerides | Lipoprotein Lipase, | Ferricyanide or | Phenazine Methosulfate |
| | Glycerol Kinase, and | Phenazine | |
| | Glycerol-3-Phosphate | Ethosulfate | |
| | Oxidase | | |
| Lactate | Lactate Oxidase | Ferricyanide | 2,6-Dichloro-1,4- |
| | | | Benzoquinone |
| Lactate | Lactate Dehydrogenase | Ferricyanide | |
| | and Diaphorase | Phenazine | |
| | | Ethosulfate, or | |
| | | Phenazine | |
| | | Methosulfate | |
| Lactate | Diaphorase | Ferricyanide | Phenazine Ethosulfate, or |
| Dehydrogenase | | | Phenazine Methosulfate |
| Pyruvate | Pyruvate Oxidase | Ferricyanide | |
| Alcohol | Alcohol Oxidase | Phenylenediamine | |
| Bilirubin | Bilirubin Oxidase | 1-Methoxy- | |
| | | Phenazine | |
| | | Methosulfate | |
| Uric Acid | Uricase | Ferricyanide | |

This table is disclosed in U.S. Pat. No. 6,755,949 and incorporated herein by reference.

The cover 150 is disposed on the insulating spacing layer 130 and covers the opening 131. In this embodiment, the first connection region x₁ is exposed by the cover 150, while the second connection region x₃ is covered by the cover 150. A sampling space (i.e. reaction area) with capillary attraction is formed between the insulating substrate 110 and the cover 150, which allows sample to enter into the reaction area in the direction indicated by the arrow shown in FIG. 1. If the area of the sampling space is fixed, its volume depends on the thickness of the insulating spacing layer 130. Generally, the thickness of the insulating spacing layer 130 is between 0.005 and 0.3 millimeter, but not limited thereto. In another embodiment, the present invention can further include a partition layer (not shown) disposed between the insulating spacing layer 130 and the cover 150, such that the volume of the sampling space is decided by the sum of thicknesses of the partition layer and the insulating spacing layer 130.

The cover 150 of the present invention can be made of transparent or translucent material, so that the users can check whether the sample has been disposed within the opening 131 (i.e. the reaction area) in order to avoid a false result. Further, the lower surface of the cover 150 close to the reaction area can be coated with a hydrophile material to enhance the capillary action along the inner surface of the reaction area, whereby the sample can be conducted into the reaction area more quickly and efficiently. The cover 150 further includes a vent 151 corresponding to the opening 131 for expelling the air inside the reaction area to further enhance the capillary action. Generally, the vent 151 is near the inner end of the opening 131. The shape of the vent 151 is not limited by the present invention, and can be, for example, circle, ellipse, rectangle, a rhombus, etc.

The identifying unit 160 is disposed in the second connection region x₃ and includes a plurality of electronic elements a1, a2, a3, and a4. The electronic elements a1-a4 can be implemented by any electrically conductive elements, such as an electrical passive element. In one embodiment, the electronic elements a1-a4 can be a resistor formed of the same material as that of the set of electrodes 120. In another embodiment, the electronic elements of the identifying unit 160 can include a resistor, a capacitor, an inductor and/or the combination thereof. When the biochemical test strip 100 is inserted into a measurement device, the measurement device can detect the number and the location of the electronic elements a1-a4 of the identifying unit 160, whereby the measurement device can recognize the type of the biochemical test strip 100 and select corresponding correction parameter and/or test mode for executing the test procedure. In other words, an identification code of the biochemical test strip 100 can be determined by number and location of the plurality of electronic elements a1-a4, which enables a measurement device to recognize the type of the biochemical test strip 100. Typically, as long as the identifying unit 160 is located in the second connection region x₃, the present invention doesn't limit the arrangement, shape, and the number of the electronic elements of the identifying unit 160.

In the embodiment shown in FIG. 1, the identifying unit 160 is formed on an upper surface of the cover 150 by a screen printing method, an imprinting method, a thermal transfer printing method, a spin coating method, or an ink-jet printing method.

FIG. 2 illustrates an explosive view of a biochemical test strip 200 according to another embodiment of the present invention, which includes an insulating substrate 210, a set of electrodes 220, an insulating spacing layer 230, a reaction layer 240, and a cover 250. The set of electrodes 220 includes a working electrode 221, a reference electrode 222, and a sensing electrode 223. The insulating spacing layer 230 includes an opening 231 for accommodating the reaction layer 240. The cover 250 includes a vent 251 corresponding to the opening 231. Furthermore, a first connection region y₁, a sensing region y₂, and a second connection region y₃ can be defined in the biochemical test strip 200 based on their respective functions. The functions and structures of above components of the biochemical test strip 200 are same as that shown in FIG. 1, so the detail description thereof is omitted.

The biochemical test strip 200 further includes an insulating slice 270 and an identifying unit 260 formed on the insulating slice 270. The insulating slice 270 is disposed in the second connection region y₃ and above the cover 250, and can be made of PVC insulating tape, PET insulating tape, or other insulating material. Both of the insulating spacing layer 230 and the cover 250 cover the second connection region y₃, and therefore the insulating slice 270 covers a part of the cover 250. In the embodiment shown in FIG. 2, the identifying unit 260 includes five electronic elements b1, b2, b3, b4, and b5, which are formed on the surface of the insulating slice 270 by screen printing method, imprinting method, thermal transfer printing method, spin coating method, or ink-jet printing method. Actually, the number and location of the electronic elements of the identifying unit 260 can be programmed to set an identification code of the biochemical test strip 200. Therefore, after determining the batch or the type of the biochemical test strip 200, the insulating slice 270 having the corresponding identifying unit 260 can be disposed on the cover 250 by adhesion or other similar method, so as to enables a measurement device to recognize the type of the biochemical test strip 200.

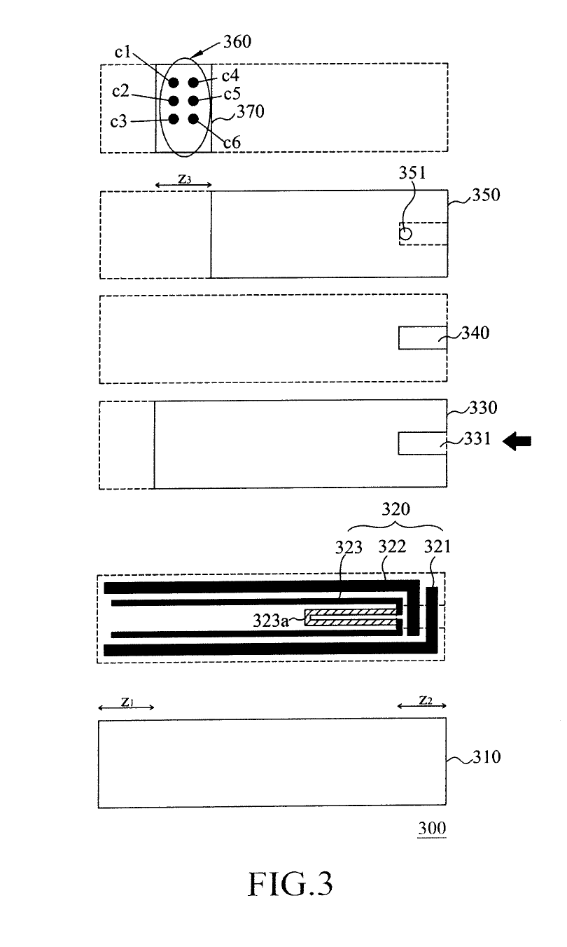

FIG. 3 illustrates an explosive view of a biochemical test strip 300 according to another embodiment of the present invention, which includes an insulating substrate 310, a set of electrodes 320, an insulating spacing layer 330, a reaction layer 340, a cover 350, an insulating slice 370, and an identifying unit 360 formed on the insulating slice 370. The set of electrodes 320 includes a working electrode 321, a reference electrode 322, and a sensing electrode 323. The insulating spacing layer 330 includes an opening 331 for accommodating the reaction layer 340. The cover 350 includes a vent 351 corresponding to the opening 331. Furthermore, a first connection region z₁, a sensing region z₂, and a second connection region z₃ can be defined in the biochemical test strip 300 based on their respective functions. The functions and structures of above components of the biochemical test strip 300 are same as that shown in FIG. 2, so the detail description thereof is omitted. In this embodiment, the identifying unit 360 includes six electronic elements c1, c2, c3, c4, c5, and c6. Different from the cover 250 of the biochemical test strip 200 shown in FIG. 2, the cover 350 of the biochemical test strip 300 exposes both of the first connection region z₁ and the second connection region z₃, and the insulating spacing layer 330 covers the second connection region z₃, such that the insulating slice 370 contacts a part of the insulating spacing layer 330 without covering the cover 350.

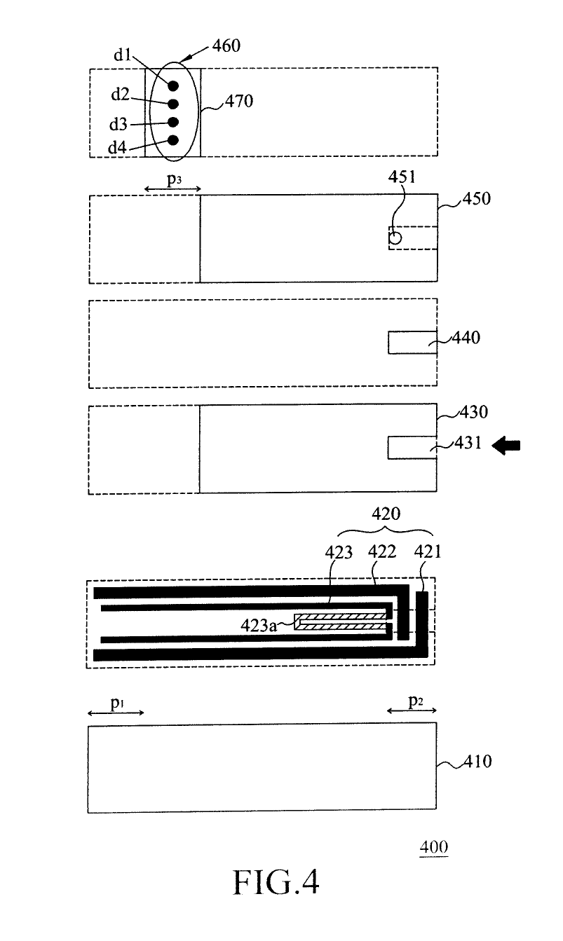

FIG. 4 illustrates an explosive view of a biochemical test strip 400 according to another embodiment of the present invention, which includes an insulating substrate 410, a set of electrodes 420, an insulating spacing layer 430, a reaction layer 440, a cover 450, an insulating slice 470, and an identifying unit 460 formed on the insulating slice 470. The set of electrodes 420 includes a working electrode 421, a reference electrode 422, and a sensing electrode 423. The insulating spacing layer 430 includes an opening 431 for accommodating the reaction layer 440. The cover 450 includes a vent 451 corresponding to the opening 431. Furthermore, a first connection region p1, a sensing region p2, and a second connection region p3 can be defined in the biochemical test strip 400 based on their respective functions. The functions and structures of above components of the biochemical test strip 400 are same as that shown in FIG. 3, so the detail description thereof is omitted. In this embodiment, the identifying unit 460 includes four electronic elements d1, d2, d3, and d4. Different from the biochemical test strips 200 and 300 shown in FIGS. 2 and 3, both of the first connection region p1 and the second connection region p3 are exposed by both of the insulating spacing layer 430 and the cover 450, such that the insulating slice 470 contacts the insulating substrate 410 and a part of the set of electrodes 420 without covering the insulating spacing layer 430 and the cover 450.

It can be seen from the above embodiments, the distance between the identifying unit and the upper surface of the insulating substrate is different from the distance between the set of electrodes and the upper surface of the insulating substrate. In the embodiment shown in FIG. 1, the height difference between the identifying unit 160 and the set of electrodes 120 is approximately equal to the sum of thicknesses of the insulating spacing layer 130 and the cover 150. In the embodiment shown in FIG. 2, the height difference between the identifying unit 260 and the set of electrodes 220 is approximately equal to the sum of thicknesses of the insulating spacing layer 230, the cover 250, and the insulating slice 270. In the embodiment shown in FIG. 3, the height difference between the identifying unit 360 and the set of electrodes 320 is approximately equal to the sum of thicknesses of the insulating spacing layer 330 and the insulating slice 370. In the embodiment shown in FIG. 4, the height difference between the identifying unit 460 and the set of electrodes 420 is approximately equal to the thickness of the insulating slice 470. Furthermore, it should be noted that although the identifying unit and the set of electrodes shown in each of FIGS. 1-4 are located on the same side of the insulating substrate (i.e. disposed on the same surface), but the present invention is not limited to this only. For example, the identifying unit can be disposed on one surface of the insulating substrate, and the set of electrodes can be disposed on another opposite surface. In addition, it should be noted that the shape and the location of the first connection region, the second connection region, and the sensing region are not limited by the present invention, as long as each of the first connection region and the second connection region can be electrically connected to the measurement device, and the reaction region can be accommodated by the sensing region.

FIG. 5 is a block diagram of a measurement device 500 according to an embodiment of the present invention, which can be used with each of the biochemical test strips 100-400 shown in FIGS. 1-4. The measurement device 500 includes a connector 510 and a microprocessor 520 coupled to the connector 510. The connector 510 includes a first measurement region 512 and a second measurement region 514 respectively corresponding to the first connection region and the second connection region described-above. The first measurement region 512 includes a plurality of connecting terminals respectively corresponding to the electrodes of the set of electrodes, and the second measurement region 514 includes a plurality of connecting terminals respectively corresponding to the plurality of electronic elements of the identifying unit. Therefore, the biochemical test strip can be electrically coupled with the measurement device 500 through the connector 510. The digital data 525, which can be correction parameters, test modes or other similar data, are stored in the microprocessor 520. Since the electronic elements of the identifying units of the biochemical test strips 100-400 shown in FIGS. 1-4 are different in locations and numbers, there are numerous possible configurations of the electrical connection between the second measurement region 514 of the connector 510 and the identifying units. When a biochemical test strip is inserted into the measurement device 500, a signal corresponding to the electrical connection between the biochemical test strip and the measurement device 500 can be generated and transmitted to the microprocessor 520. After receiving the signal, the microprocessor 520 can select corresponding correction parameter or test mode from the digital data 525 to execute the measurement procedure. In other words, the electrical characteristic of the identifying unit can be changed by varying the number and the location of the electronic elements of the identifying unites, so as to allow the measurement device to recognize the type of the biochemical test strip.

Referring to FIG. 5 again, the measurement device 500 further includes a monitor 530 for displaying the measurement result and a power source 540 for supplying power. In another embodiment, the monitor 530 and the power source 540 can be external devices, not included within the measurement device 500.

FIGS. 6A, 6B, 7A, 7B, 8, 9, 10A and 10B are illustrative diagrams showing the insulating slices and the electronic elements thereon according to different embodiments of the present invention respectively. Referring to FIGS. 6A and 6B, the insulating slice 670 has four electronic elements e1, e2, e3, e4 and the insulating slice 675 has two electronic elements f1, f2, while both of the insulating slices 670 and 675 are corresponding to the same connector. In one embodiment, the connector corresponding to the insulating slices 670 and 675 includes at least four connecting terminals respectively corresponding to the electronic elements e1-e4. When a biochemical test strip having the insulating slice 670 is inserted into the measurement device, the four connecting terminals of the connector can be electrically connected with electronic elements e1-e4. On the other hand, when a biochemical test strip having the insulating slice 675 is inserted into the measurement device, only two connecting terminals of the connector can be electrically connected with electronic elements f1 and f2, but the other two connecting terminals of the connector will remain in open circuit position. Therefore, the measurement device can identify the type of the biochemical test strip by detecting electrical connection between the connecting terminals of the connector and the electronic elements formed on the insulating slice. In other words, the number and the location of the electronic elements on the insulating slice can be adjusted by the designer according to practical applications to establish the desired electrical connection with the connector of the measurement device, which is indicative of a specific identification code of the biochemical test strip.

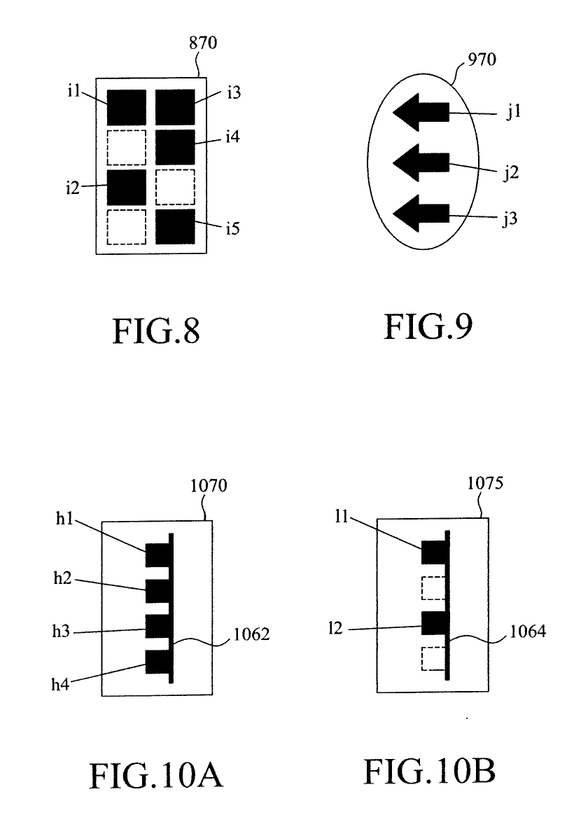

Referring to FIGS. 7A and 7B, the insulating slice 770 has eight electronic elements g1-g8 and the insulating slice 775 has six electronic elements h1-h6, while both of the insulating slices 770 and 775 are corresponding to the same connector. As described above, the electrical connections from the connector to the insulating slices 770 and 775 are different, whereby the measurement device can recognize the type of the biochemical test strip. FIG. 8 illustrates an insulating slice 870 according to another embodiment of the present invention, which includes five square-shaped electronic elements i1-i5. FIG. 9 illustrates an insulating slice 970 according to another embodiment of the present invention, which includes three electronic elements j1-j3. In the embodiment shown in FIG. 9, the insulating slice 970 has an elliptic shape, and each of the electronic elements j1-j3 has an arrow shape. It can be seen from the above embodiments, the identifying unit of the present invention can include the electronic elements of various shapes, such as straight shape, rectangular shape, polygonal shape, wave shape, arc shape, circular shape, or <-like shape. Furthermore, the shape of the insulating slice can be, but not limited to, square shape, rectangular shape, circular shape, elliptic shape, or irregular shape. Next, referring to FIGS. 10A and 10B, the insulating slice 1070 has four electronic elements h1-h4 and a linking unit 1062 and the insulating slice 1075 has two electronic elements l1-l2 and a linking unit 1064, while both of the insulating slices 1070 and 1075 are corresponding to the same connector. One side of the linking unit 1062 is connected to one end of each of the electronic elements h1-h4 for providing a common ground for these four electronic elements. Similarly, one side of the linking unit 1064 is connected to one end of each of the electronic elements l1-l4 for providing a common ground for these two electronic elements. It should be noted that each of the biochemical test strips shown in FIGS. 1-4 can further include a linking unit for providing a common ground for the components formed on the strip.

According to one aspect of the present invention, after a biochemical test strip has been made, the identifying unit can be formed on the cover or the insulating slice with the identifying unit can be attached to the test strip according to the batch, characteristic or function of the biochemical test strip, so that the measurement device can recognize the type of the biochemical test strip and then select the corresponding correction parameters, test modes, or other information, which are stored in advance in the measurement device, to perform the measurement procedure. To sum up, the goal of avoiding the use of code card, reducing the production cost, reducing the possibility of man-made errors, and increasing operating convenience can be achieved by the present invention.

The above illustration is for preferred embodiments of the present invention, is not limited to the claims of the present invention. Equivalent amendments and modifications without departing from the spirit of the invention should be included in the scope of the following claims.

What is claimed is:

1. A biochemical test strip having a first connection region, a second connection region and a sensing region defined thereon, the biochemical test strip comprising:
an insulating substrate;
a set of electrodes disposed on the insulating substrate, wherein one end of the set of electrodes is in the first connection region;
an insulating slice disposed on the set of the electrodes and exposing at least the first connection region; and
an identifying unit formed on a surface of the insulating slice in the second connection region, wherein the second connection region is different from the first connection region;
wherein the identifying unit comprises a plurality of electronic elements and an identification code of the biochemical test strip is determined by number and location of the plurality of electronic elements.

2. The biochemical test strip according to claim 1, wherein each of the plurality of electronic elements is a passive element.

3. The biochemical test strip according to claim 1, wherein the identifying unit is formed on the surface of the insulating slice by a screen printing method, an imprinting method, a thermal transfer printing method, a spin coating method, or an ink-jet printing method.

4. The biochemical test strip according to claim 1, wherein the insulating slice is made of polyvinylchloride (PVC) insulating tape, polyethylene terephthalate (PET) insulating tape, or other insulating material.

5. The biochemical test strip according to claim 1, further comprising:
an insulating spacing layer covering a part of the insulating substrate, wherein the first connection region and a part of the sensing region are exposed by the insulating spacing layer, and the exposed part of the sensing region defines a reaction area; and
a reaction layer disposed in the reaction area, wherein the reaction layer comprises an oxidoreductase.

6. The biochemical test strip according to claim 5, wherein the insulating slice is disposed on the insulating spacing layer and covers the reaction area, the insulating slice having a vent corresponding to the reaction area.

7. The biochemical test strip according to claim 5, further comprising a cover disposed on the insulating spacing layer and covering the reaction area, wherein the cover exposes the first connection region, and the insulating slice covers a part of the cover.

8. The biochemical test strip according to claim 5, further comprising a cover disposed on the insulating spacing layer and covering the reaction area, wherein the cover exposes the first connection region and the second connection region, and the insulating slice contacts a part of the insulating spacing layer.

9. The biochemical test strip according to claim 5, further comprising a cover disposed on the insulating spacing layer and covering the reaction area, wherein the insulating spacing layer exposes the second connection region, the cover exposes the first connection region and the second connection region, and the insulating slice contacts a part of the insulating substrate.

10. The biochemical test strip according to claim 1, further comprising a linking unit formed on a surface of the insulating slice for providing a common ground, wherein one terminal of each of the plurality of electronic elements is connected to one side of the linking unit.

11. A biochemical test strip having a first connection region, a second connection region and a sensing region defined thereon, the biochemical test strip comprising:
an insulating substrate having an upper surface;
a set of electrodes disposed on the upper surface of the insulating substrate, wherein one end of the set of electrodes is in the first connection region; and
an identifying unit disposed in the second connection region, wherein a distance between the identifying unit and the upper surface is different from a distance between the set of electrodes and the upper surface, wherein the second connection region is different from the first connection region;
wherein the identifying unit comprises a plurality of electronic elements and an identification code of the biochemical test strip is determined by number and location of the plurality of electronic elements.

12. The biochemical test strip according to claim 11, wherein each of the plurality of electronic elements is a passive element.

13. The biochemical test strip according to claim 11, further comprising:
an insulating spacing layer covering a part of the insulating substrate, wherein the first connection region and a part of the sensing region are exposed by the insulating spacing layer, and the exposed part of the sensing region defines a reaction area;
a reaction layer disposed in the reaction area, wherein the reaction layer comprises an oxidoreductase; and
a cover disposed on the insulating spacing layer for covering the reaction area and exposing the first connection region, the cover having a vent corresponding to the reaction area.

14. The biochemical test strip according to claim 13, wherein the identifying unit is formed on a surface of the cover.

15. The biochemical test strip according to claim 13, further comprising an insulating slice covering a part of the cover, wherein the identifying unit is formed on a surface of the insulating slice.

16. The biochemical test strip according to claim 13, further comprising an insulating slice, wherein the identifying unit is formed on a surface of the insulating slice, the cover exposes the second connection region, and the insulating slice contacts a part of the insulating spacing layer.

17. The biochemical test strip according to claim 13, further comprising an insulating slice, wherein the identifying unit is formed on a surface of the insulating slice, the insulating spacing layer and the cover exposes the second connection region, and the insulating slice contacts a part of the insulating substrate.

18. A measurement device for use with the biochemical test strip of claim 1, the measurement device comprises:
a connector comprising a first measurement region corresponding to the set of electrodes and a second measurement region corresponding to identifying unit, the first measurement region and the second measurement region respectively electrically coupled to the set of electrodes and the identifying unit for receiving a signal corresponding the identifying unit; and
a microprocessor coupled to the connector for receiving the signal from the connector.

19. The measurement device according to claim 18, wherein a plurality of correction parameters or a plurality of test modes are stored in the microprocessor, and the microprocessor selects one of the correction parameters or one of the test mode for execution according to the received signal.

20. The measurement device according to claim 18, wherein the first measurement region comprises a plurality of connecting terminals respectively corresponding to the electrodes of the set of electrodes, and the second measurement region comprises a plurality of connecting terminals respectively corresponding to the plurality of electronic elements.

21. A biochemical test system, comprising:
the biochemical test strip of claim 1; and
a measurement device, comprising a microprocessor and a connector, wherein the connector comprises a plurality of connecting terminals respectively corresponding to the set of electrodes and the identifying unit, the plurality of connecting terminals respectively electrically coupled to the set of electrodes and the identifying unit for receiving a signal corresponding to the identifying unit, the microprocessor coupled to the connector for receiving the signal from the connector.

22. The biochemical test system according to claim 21, wherein a plurality of correction parameters or a plurality of test modes are stored in the microprocessor, and the microprocessor selects one of the correction parameters or one of the test modes for execution according to the received signal.
